(19) United States
(12) Patent Application Publication
(10) Pub. No.: US 2012/0083450 A1
(43) Pub. Date: Apr. 5, 2012

(54) CEREBROPROTEIN HYDROLYSATE FOR TREATING BRAIN INJURIES AND NEURODEGENERATIVE CONDITIONS EFFECTING THE BRAIN

(76) Inventor: Douglas James Phillips, II, Tequesta, FL (US)

(21) Appl. No.: 12/896,002

(22) Filed: Oct. 1, 2010

(51) Int. Cl. A61K 38/02; A61P 25/28

(52) U.S. Cl. 514/17.7

(57) ABSTRACT

Cerebroprotein hydrolysate is administered intravenously and/or orally to help treat a range of brain injuries and neurodegenerative conditions effecting the brain including Traumatic Brain Injury (TBI), concussions, injuries resulting from automobile accidents, sports injuries, falls that impact the head, accidents and acts of violence that impact the head and brain including injuries incurred from combat related activities, Alzheimer's disease, Parkinson's disease, Multiple Sclerosis, and other neurological conditions that adversely effect the function of the brain and central nervous system. The types of brain injuries and neurodegenerative conditions that may be treated with cerebroprotein hydrolysate range from mild to severe. Cerebroprotein hydrolysate treatment effectively assists the brain in rebuilding neurons, synapses, and neurological tissue by infusing the brain with specific proteins, neuropeptides, and amino acids to assist with brain repair.

### BACKGROUND OF THE INVENTION

Cerebroprotein hydrolysate is a brain protein often extracted and derived from an animal source consisting of amino acids and neuropeptides. Cerebroprotein hydrolysate is most often extracted through a process of enzymatic hydrolysis.

### SUMMARY OF THE INVENTION

This invention is based on using cerebroprotein hydrolysate for the treatment of brain injuries and neurodegenerative conditions ranging from mild to severe. This invention is intended to directly act on the brain to help repair and protect neurons, rebuild synaptic and other brain circuit connections, and assist the brain in recovering from injury and neurodegeneration resulting from neurological disease. Cerebroprotein hydrolysate may be administered orally in capsule or tablet form, injected into the body, and through intravenous routes of administration. This invention is intended to cover any and all routes of administration into the human body of cerebroprotein hydrolysate for the specific purposes of treating brain-related medical conditions including, but not limited to, traumatic brain injury, Alzheimer's disease, Parkinson's disease, Multiple Sclerosis, concussions, shell shock, head falls, and sports injuries effecting the brain.

What is claimed is:

1. Administration of cerebroprotein hydrolysate is an effective treatment for brain injuries and neurodegenerative conditions.

2. The process of treating brain injuries and neurodegenerative conditions with cerebroprotein hydrolysate can assist the brain repair neurons, synapses and other structures of neuroanatomy.

3. The process of improving brain functions related to memory, concentration, focus, vision, hearing, bodily movement, speech, mood, long-term planning, personality, and other neurologically relevant functions related to proper brain functioning using cerebroprotein hydrolysate.

4. The method of claim 1 wherein the mode of administration is oral, intravenous, or by injection into the body.

5. The process of claim 2 wherein the mode of administration is oral, intravenous, or by injection into the body.

6. The process of claim 3 wherein the mode of administration is oral, intravenous, or by injection into the body.
